(19) United States
(12) Patent Application Publication
(10) Pub. No.: US 2007/0105954 A1
(43) Pub. Date: May 10, 2007

(54) Formulation containing a carboxylic acid or an ester thereof

(75) Inventor: Basant Kumar Puri, Cambridge (GB)

Correspondence Address: KAPLAN GILMAN GIBSON & DERNIER L.L.P., 900 ROUTE 9 NORTH, WOODBRIDGE, NJ, 07095, US

(73) Assignee: INGENNUS LIMITED, CAMBRIDGE (GB)

(21) Appl. No.: 10/585,026

(22) PCT Filed: Dec. 31, 2004
(86) PCT No.: PCT/GB04/05461

§ 371 Date: Jan. 19, 2007

(30) Foreign Application Priority Data
Dec. 31, 2003 (GB) 0330206.4
Jul. 6, 2004 (GB) 0415097.5

(51) Int. Cl. A61K 31/215; A61K 31/202

(52) U.S. Cl. 514/529; 514/559; 514/560

(57) ABSTRACT

A formulation comprising eicosapentaenoic acid, or an ester thereof, and a triterpene, or an ester thereof, and the use of such a formulation.

This invention relates to a formulation comprising eicosapentaenoic acid, or an ester thereof, and a triterpene, or an ester thereof, and to its use in the treatment of, or manufacture of a medicament for the treatment of, a number of disorders. The formulation also has cosmetic uses. The invention also provides a method for the preparation of a formulation to be an orally administered or a method for the preparation of a formulation to be topically administered.

The present invention provides a formulation comprising:
(a) eicosapentaenoic acid or an ester thereof; and
(b) a triterpene or an ester thereof.

Eicosapentaenoic acid can be extracted in a natural form from the oil of fish, in particular from so-called ‘oily fish’ such as sardines and salmon. Alternatively, eicosapentaenoic acid can be synthesised, for example ethyl eicosapentaenoic acid. Esters of eicosapentaenoic acid may be naturally occurring or synthesised. The formulation of the present invention may contain natural eicosapentaenoic acid (such as the free fatty acid), synthetic eicosapentaenoic acid, a naturally occurring ester of eicosapentaenoic acid or a synthetic ester of eicosapentaenoic acid, or a combination thereof. Preferably the eicosapentaenoic acid is ultra pure, that is, it is substantially free of any impurities. Such impurities may include docosahexaenoic acid.

Triterpenes refer to a family of naturally occurring compounds which may also be referred to as triterpenoids. The formulation of the invention may comprise a naturally occurring triterpene, a synthetic triterpene, a naturally occurring ester of a triterpene or a synthetic ester of a triterpene, or a combination thereof. Preferably the triterpene is a 3-O-trans caffeoyl derivative of betulinic acid, morolic acid or oleanolic acid, faradiol-O-laurate, faradiol-O-palmitate or faradiol-O-myristate. Naturally occurring triterpenes can be isolated from a variety of plants including the flower heads of marigolds (Calendula officinalis), Zygophyllum eichwaldii, Carthamus lanatus, Oenothera bienni (evening primrose) or Pyrus comminus. Preferably the triterpene in a formulation according to the invention is provided in the form of evening primrose oil isolated from the evening primrose plant. Preferably the evening primrose oil is virgin evening primrose oil, which is cold-pressed and non-raffinated.

The formulation may comprise up to 99% w/w of eicosapentaenoic acid or an ester thereof. Alternatively the formulation may comprise up to 99% w/w of triterpene or an ester thereof. The formulation may comprise up to 50% w/w of eicosapentaenoic acid or an ester thereof. The formulation may comprise up to 50% w/w of triterpene or an ester thereof. The formulation may comprise up to 70% w/w of eicosapentaenoic acid or an ester thereof, more preferably of 20 to 40% w/w, and 1 to 30% w/w of a triterpene or an ester thereof.

The amount of eicosapentaenoic acid or synthetic ester thereof, and triterpene or synthetic ester thereof, required to achieve the desired therapeutic or cosmetic effect will, of course, vary depending of the compounds used, the route of administration and the disorder or condition to be treated.

Preferably the formulation comprises eicosapentaenoic acid or an ester thereof, and a triterpene or an ester thereof in a pharmaceutically acceptable form.

The formulation may also comprise a pharmaceutical carrier, diluent or excipient.

The formulation may also comprise one or more of a lubricant, a flavouring, a taste masking agent, a fragrance and a preservative.

Formulations containing eicosapentaenoic acid or an ester thereof, and a triterpene or an ester thereof, may also include other compounds for co-administration. In one embodiment such compounds may include gamma-linolenic acid and docosahexaenoic acid. In an alternative embodiment the formulation does not contain the compound docosahexaenoic acid, or is substantially free of docosahexaenoic acid. Wherein substantially free means that there is less than about 0.1% docosahexaenoic acid in the formulation, preferably there is less than about 0.01% docosahexaenoic acid and more preferably less than about 0.001% docosahexaenoic acid in the formulation. It is considered that in some circumstances docosahexaenoic acid can inhibit some of the benefits of eicosapentaenoic acid or an ester thereof. Known compositions or formulations containing eicosapentaenoic acid, such as fish oils, also contain docosahexaenoic acid. In order to obtain eicosapentaenoic acid which is free or substantially free of docosahexaenoic acid from fish oil, the eicosapentaenoic acid must be extracted from the fish oil.

The formulation may also comprise conjugated linoleic acid. Preferably the formulation contains between about 0.1% and about 25% w/w conjugated linoleic acid, more preferably the formulation comprises between about 1% and about 15% conjugated linoleic acid. More preferably, the formulation comprises between about 10% and about 15% conjugated linoleic acid. The presence of conjugated linoleic acid may improve the efficacy of the formulation according to the invention in the treatment of a variety of physiological and disease states, including those listed below.

The formulation comprising eicosapentaenoic acid or an ester thereof, and a triterpene or an ester thereof, may be used to treat a variety of physiological and disease states including rheumatoid arthritis, osteoarthritis, back-ache, psoriasis, pre-menstrual syndrome, bacterial infections, viral infections, fatigue, such as chronic fatigue syndrome, insomnia, anxiety, obesity, influenza, diabetes mellitus, alcoholism, cancer, neurological disorders such as multiple sclerosis, epilepsy, tardive dyskinesia and choreiform disorders such as Huntington's disease, psychiatric disorders such as depression and attention-deficit/hyperactivity disorder, cardiovascular disorders such as hyperlipidemia and high blood pressure, dermatological disorders such as eczema and atopic dermatitis, respiratory disorders, learning disabilities and ageing.

Many of the above medical conditions have a final common pathway that involves inflammation, for example myocardial infarction (heart attacks), sudden death from cardiovascular causes, stroke, rheumatoid arthritis, asthma, skin disorders such as psoriasis, inflammatory bowel disorders, and cerebral disorders such as Alzheimer's disease. It is believed that the formulation of the present invention has powerful anti-inflammatory and immuno-modulating effects and thus can be used to treat the above plethora of medical conditions.

The formulation comprising eicosapentaenoic acid or an ester thereof, and a triterpene or an ester thereof, may be administered orally.

The formulation may be administered orally as a liquid, a paste, a tablet or a capsule.

Preferably a capsule for oral administration contains a formulation comprising between about 260 mg and about 300 mg of eicosapentaenoic acid and between about 80 mg and about 120 mg of virgin evening primrose oil, and preferably substantially none, or no, docosahexaenoic acid. More preferably a capsule contains a formulation comprising about 280 mg of eicosapentaenoic acid, about 100 mg of virgin evening primrose oil and preferably substantially none, or no, docosahexaenoic acid. The capsule may also comprise between about 3 mg and about 100 mg of conjugated linoleic acid, more preferably between about 5 mg and about 80 mg of conjugated linoleic acid, preferably about 60 mg of conjugated linoleic acid.

The oral formulation may be prepared as an inert porous matrix tablet which is obtained by mixing the eicosapentaenoic acid or an ester thereof, and a triterpene or an ester thereof, with waxes or water insoluble polymers and with fillers and binders. Paraffin, polyvinylchloride, ethylcellulose, stearylic alcohol, cetylic alcohol, carnauba wax, polyethylene, polyvinyl acetate, polymethyl methacrylate could be used as suitable diffusion retarding compounds. Other excipients used in the preparation of such tablets may include lactose, mannitol, calcium phosphate, magnesium stearate, hydroxypropyl methylcellulose, methyl cellulose, polyvinylpyrrolidone, aluminium silicate, sodium carbonate, potassium phosphate or other suitable materials.

Alternatively, the formulation comprising eicosapentaenoic acid or an ester thereof, and a triterpene or an ester thereof, may be administered topically. The formulation to be applied topically may also comprise one or more of occlusive agent, a surfactant system, a solvent and water.

One or more various solvents that may be present in the topical formulation comprise various short-chain alcohols including, but not limited to, ethyl alcohol, propylene alcohol, triacetin, hexylen glycol and combinations thereof. The solvent may be present in an amount ranging from about 5.0 to about 30.0 w/w %.

Suitable occlusive agents that may be present in the topical formulation include, but are not limited to, petrolatum, microcrystalline wax, dimethicone, beeswax, mineral oil, squalane, liquid paraffin, shea butter, carnauba wax, SEPIGELO™ (a blend ofisoparaffin/polyacrylamide/

laureth-7), and combinations thereof. The occlusive agent may be present in an amount of at least about 10.1 w/w %.

Suitable surfactant systems comprise at least one surfactant and exhibit a HLB value in a range from about 7.0 to about 10.9. The surfactant system may be present in the formulation in an amount ranging from about 0.25 to about 10.0 w/w%. Suitable surfactants include, but are not limited to, CETOMACROGOLO™ 1000 (Crodor, Inc.), glycerol monostearate, glycerol distearate, glyceryl stearate, polyoxyethylene stearate, a blend of glyceryl stearate and PEG-100 stearate (as ARLACEL™ 165), polysorbate 40, polysorbate 60, polysorbate 80, CETETH™-200, sorbitan monopalimate, sorbitan monostearate, sorbitan monooleate, and combinations thereof.

The topical formulation may also include a carrier, a skin conditioner, a preservative, a buffer, a fragrance, water or combinations thereof.

According to another aspect the invention provides a method for the treatment of various physiological and disease states including rheumatoid arthritis, osteoarthritis, back-ache, psoriasis, pre-menstrual syndrome, bacterial infections, viral infections, fatigue, such as chronic fatigue syndrome, insomnia, anxiety, obesity, influenza, diabetes mellitus, alcoholism, cancer, neurological disorders such as multiple sclerosis, epilepsy, tardive dyskinesia and choreiform disorders such as Huntington's disease, psychiatric disorders such as depression and attention-deficit/hyperactivity disorder, cardiovascular disorders such as hyperlipidemia and high blood pressure, dermatological disorders such as eczema and atopic dermatitis, respiratory disorders, learning disability and ageing, in a subject comprising administering to the subject an effective amount of a formulation comprising eicosapentaenoic acid or an ester thereof, and a triterpene or an ester thereof.

Preferably the formulation comprises substantially no docosahexaenoic acid. Preferably the formulation comprises no docosahexaenoic acid.

According to a further aspect the invention provides a formulation comprising eicosapentaenoic acid or an ester thereof, and a triterpene or an ester thereof for use in a method of treatment of a human or animal body by surgery or therapy or of diagnosis practised on the human or animal body.

In a further aspect the invention provides the use of eicosapentaenoic acid or an ester thereof, and a triterpene or an ester thereof, in the manufacture or preparation of a medicament for the treatment of various physiological and disease states including rheumatoid arthritis, osteoarthritis, back-ache, psoriasis, pre-menstrual syndrome, bacterial infections, viral infections, fatigue, such as chronic fatigue syndrome, insomnia, anxiety, obesity, influenza, diabetes mellitus, alcoholism, cancer, neurological disorders such as multiple sclerosis, epilepsy, tardive dyskinesia and choreiform disorders such as Huntington's disease, psychiatric disorders such as depression and attention-deficit/hyperactivity disorder, cardiovascular disorders such as hyperlipidemia and high blood pressure, dermatological disorders such as eczema and atopic dermatitis, respiratory disorders, learning disabilities and ageing.

Preferably the medicament comprises substantially no docosahexaenoic acid. Preferably the medicament comprises no docosahexaenoic acid.

Formulations comprising eicosapentaenoic acid or an ester thereof, and a triterpene or an ester thereof, may be used in cosmetic treatments. The cosmetic treatment may have an anti-ageing effect or reverse the process of ageing.

Preferably the formulation comprises eicosapentaenoic acid or an ester thereof, and a triterpene or an ester thereof in a cosmetically acceptable form.

Preferably the formulation comprises substantially no docosahexaenoic acid. Preferably the formulation comprises no docosahexaenoic acid.

The cosmetically acceptable formulation may also comprise a cosmetic carrier, diluent or excipient.

According to a yet further aspect the invention provides a method of cosmetic treatment comprising administering an effective amount of a formulation comprising eicosapentaenoic acid or an synthetic ester thereof, and a triterpene or an ester thereof.

Preferably, the formulation is administered as an anti-ageing formulation or to reverse the ageing process.

The cosmetic formulation may be administered orally or topically.

A yet further aspect of the invention provides a method for preparing a topical formulation comprising mixing eicosapentaenoic acid or an ester thereof and a triterpene or an ester thereof with a topically acceptable carrier.

The method may also comprise mixing the eicosapentaenoic acid or an ester thereof and the triterpene or an ester thereof with one or more of the following a solvent, an occlusive agent, a surfactant system and water.

The method may also comprise mixing the eicosapentaenoic acid or an ester thereof and the triterpene or an ester thereof with one or more of vitamin E (natural or an analogue), an emulsifying wax, honey, water, fragrance, an emulsifier and a mixture of ethyl, propyl and butyl parabens.

A still further aspect of the invention provides a method for preparing an orally administered formulation comprising mixing eicosapentaenoic acid or an ester thereof and a triterpene or an ester thereof with an orally acceptable carrier.

The method may also include mixing vitamin E (natural or an analogue) into the formulation. Vitamin E is an antioxidant and thus helps prevent unwanted oxidation.

The method may also include adding a flavouring or a taste masking agent to the formulation.

It will be appreciated that the compounds of eicosapentaenoic acid or an ester thereof, and a triterpene or an ester thereof, may be administered simultaneously, either in the same or different formulations, or sequentially. When there is sequential administration, the delay in administering the second and any subsequent active ingredient should not be such as to lose the beneficial therapeutic or cosmetic effect of the combination. In a preferred aspect of the invention the eicosapentaenoic acid or an ester thereof, and the triterpene or an ester thereof, are administered in a combined formulation.

According to a further aspect the invention provides a method for the treatment of various physiological and disease states including rheumatoid arthritis, osteoarthritis, back-ache, psoriasis, pre-menstrual syndrome, bacterial infections, viral infections, fatigue, such as chronic fatigue syndrome, insomnia, anxiety, obesity, influenza, diabetes mellitus, alcoholism, cancer, neurological disorders such as multiple sclerosis, epilepsy, tardive dyskinesia and choreiform disorders such as Huntington's disease, psychiatric disorders such as depression and attention-deficit/hyperactivity disorder, cardiovascular disorders such as hyperlipidemia and high blood pressure, dermatological disorders such as eczema and atopic dermatitis, respiratory disorders, learning disabilities and ageing, in a subject comprising administering to the subject an effective amount of eicosapentaenoic acid or an ester thereof, and a triterpene or an ester thereof, wherein the eicosapentaenoic acid, or an ester thereof, and the triterpene, or an ester thereof, are administered simultaneously, either in the same or different formulations, or sequentially.

According to a yet further aspect the invention provides the use of eicosapentaenoic acid or an ester thereof, and a triterpene or an ester thereof, administered simultaneously, either in the same or different formulations, or sequentially, in a method of treatment of a human or animal body by surgery or therapy or of diagnosis practised on the human or animal body.

It will be appreciated that preferred features of the invention discussed with reference to only some aspects of the invention can equally be applied to all aspects of the invention.

The present invention will now be illustrated, merely by way of example, with reference to the following methods and examples.

### METHOD 1

### Method of Extracting Eicosapentaenoic Acid and Triterpenes

A method of extracting eicosapentaenoic acid from fish oil is described in Enzyme Microb Technol. 2000 Apr 1;26(7):516-529. By using this method eicosapentaenoic acid is extracted substantially free of docosahexaenoic acid or with no docosahexaenoic acid.

A method of extracting triterpenes from marigolds is described in Fitoterapia. 2003 June; 74(4):328-38. More specifically this paper discloses a method for the purification of the triterpenoid esters faradiol 3-O-laurate, palmitate and myristate from the flower heads of the medicinal plant Calendula officinalis (marigold).

### METHOD 2

### Method of Preparing a Cream Formulation for Topical Administration

A method for the preparation of a cream for topical application comprising eicosapentaenoic acid and a triterpene comprises placing the following components in a receptacle at room temperature:
122 g pure eicosapentaenoic acid;
20 g pure gamma-linolenic acid;
65 g organic, virgin, cold-pressed, non-raffinated evening primrose oil (which provides the triterpene);
3.4 g D alpha tocopheryl acetate;
180 g emulsifying wax; and
48 g clear honey.

The components are stirred together and then heated for one minute.

To this mixture is then added:
540 g water;
1.5 g fragrance (e.g. citrus: lime or Lemon);
12 g of an emulsifier (to form a stable emulsion); and
30 g of a mixture of ethyl, propyl and butyl parabens.

The whole mixture is then gently stirred and heated for a further four minutes. It is then stirred slowly for a further five minutes until it has the required consistency for the cream. It is then transferred into glass jars that have been sterilized (at over 100 degrees C.) using implements that have also been sterilized. Finally, lids that have also been sterilized are fastened on to the jars, which are then left to cool.

### METHOD 3

### Method of Preparing a Formulation for Oral Administration

A method for the preparation of a formulation for oral administration containing eicosapentaenoic acid or a synthetic ester thereof, and a triterpene or a synthetic ester thereof, comprises placing the following components in a mixing bowl and manually mixing together for five minutes:
pure eicosapentaenoic acid;
pure gamma-linolenic acid;
organic, virgin, cold-pressed, non-raffinated evening primrose oil; and
D alpha tocopheryl acetate; in a ratio, by mass, of 186 to 20 to 50 to 3.2.

### METHOD 4

### Alternative Method of Preparing a Formulation for Oral Administration

An alternative method for the preparation of a formulation for oral administration containing eicosapentaenoic acid or a synthetic ester thereof, and a triterpene or a synthetic ester thereof, comprises placing the following components in a mixing bowl and manually mixing together for five minutes:
pure eicosapentaenoic acid, with no docosahexaenoic acid;
virgin, cold-pressed, non-raffinated evening primrose oil;
short chain fatty acids; and
conjugated linoleic acid. in a ratio by weight of 56:20:1:23

The resultant mixture is then used to form capsules containing 500 mg of formulation, each capsule containing a formulation comprising:
280 mg pure eicosapentaenoic acid;
100 mg virgin evening primrose oil;
5 mg conjugated linoleic acid; and
115 mg of short chain fatty acids.

The formulation comprises no docosahexaenoic acid.

Capsules containing the formulation as described above are made using by standard techniques and protocols well known to the man skilled in the art.

Case Studies on the Use of a Cream Made According to the Above Described Method 2

A number of studies have been undertaken to demonstrate the therapeutic and cosmetic effects of the cream made by the above-described Method 2.

More specifically:

Cosmetic Effect—Anti-ageing

Four subjects have thus far specifically used the cream made by the above-described Method 2 for its anti-ageing properties.
Subject 1—A female, aged 50, used the cream topically on her face and observed that within one week her skin looked younger and ‘healthier, fresher, with a radiant look’. She described the cream as being far better than anything she has ever bought (e.g. evening primrose cream). She had sensitive skin, and noticed no adverse side-effects at all.
Subject 2—a female, aged 20, used the cream topically on her face, she also had sensitive skin, and again noticed within one week no adverse side-effects at all. She described her skin as looking healthier.
Subject 3—a female, aged 51, used the cream topically on her face, this subject derived similar benefits to subject 1, and described the result as being similar to ‘botox without needles’.
Subject 4—a male aged 52, used the cream topically on his face, the subject described the effects within one week as being ‘like a face-lift without surgery’.

All four subjects asked to continue applying the cream to their faces. The female subjects wish to use it instead of a traditional cosmetic foundation application.

These initial test results demonstrate the anti-ageing cosmetic effect of a cream according to the present invention.

Therapeutic Effect—Back-ache

A female subject aged 75 with previously intractable back-ache began to derive relief of her back pain after three days' topical application of a cream made by the above-described Method 2.

Therapeutic Effect—Arthritis

Subject 1—A female aged 69 suffering from severe rheumatoid arthritis in her hands, which had not responded to traditional medical treatment, applied cream made by the above-described Method 2 to her hands and an improvement was seen within one week.
Subject 2-An 89-year-old female with severe osteoarthritis in the hands, which had never previously responded to any treatment, showed improvement after one week when applying cream, made by the above-described Method 2, to her hands. The improvements observed included relief from the pain, for the first time, and a decrease in the size of tophi (swellings).
   Therapeutic/Cosmetic Effect—Skin Sores

A 69-year-old female subject with severe rheumatoid arthritis (see above) also noticed that her skin sores on her hands were much better seven to eight days after beginning use of the cream made by the above-described Method 2. They had previously failed to respond to medical treatment and had had to be bandaged.

Therapeutic Effect—Psoriasis

Subject 1—A female aged 17 with severe intractable treatment-resistant psoriasis started to improve after two to three days following topical application of cream, made by the above-described Method 2, to her arms and legs.
Subject 2—A female aged 31 with severe psoriasis affecting her upper limbs responded after six to seven days when applying the cream, made by the above-described Method 2, to her upper limbs; she had previously tried a wide range of medical and ‘alternative’ treatments, to no avail.
   Therapeutic/Cosmetic Effect—Eczema

A 52-year-old female subject with severe eczema responded within one week to the topical application of cream, made by the above-described Method 2, again where conventional medical treatment had previously failed.

Oral Administration—Learning Difficulties

An 11-year-old boy with learning difficulties started taking 1.5 g daily of the oral formulation discussed above with reference to Method 3. Within four weeks he started to show signs of improvement according to his parents and teachers. This improvement was in several domains, including cognitive functioning, reading and understanding his school work. The improvement continued and he reached a new, higher, level of intellectual functioning after three months, which has continued to be sustained for 6 months.

Oral Administration with Capsules According to Method 4

In the following examples all patients were administered capsules containing a formulation comprising 280 mg pure eicosapentaenoic acid, 100 mg virgin evening primrose oil, 5 mg conjugated linoleic acid and no docosahexaenoic acid.

Chronic Fatigue Syndrome (Myalgic Encephalomyelitis)

By the end of August 2004, a total of 109 patients meeting the 1994 CDC Revised Diagnostic Criteria for chronic fatigue syndrome had been treated with capsules according to Method 4. The first eight were treated with a dose of between four and six capsules daily; five of them showed significant clinical improvement within three months. The remaining 101 patients were treated with eight capsules daily; 81 showed significant clinic improvements in 4 months. Improvements were particularly noticeable in the following areas: reduced fatigue; increased energy levels; improved quantity and quality of sleep; and reduced myalgia. These 81 patients no longer fulfil the 1994 CDC Revised Diagnostic Criteria for chronic fatigue syndrome.

Depression

By the end of August 2004, a total of 86 patients meeting the American Psychiatric Association DSM-IV-TR criteria for major depressive disorder had been treated with capsules according to Method 4 at an average dose of eight capsules daily. Seventy-two of had shown significant clinical improvement in 4 months. Improvements were particularly noticeable in the following areas: improved mood; improved quantity and quality of sleep; reduced fatigue; increased energy levels; reduced social phobia; reduced tearfulness; and increased libido. Suicidal feelings were amongst the first symptoms to improve—in fact, in all cases they cleared up completely. These 72 patients no longer fulfil the DSM-IV-TR criteria for major depressive disorder, and have shown marked improvements in their depression ratings as measured by the Hamilton Depression Rating Scale and the Montgomery and Asberg Depression Rating Scale.

Huntington's Disease

Two patients with Huntington's disease took capsules according to Method 4, at a dose of eight capsules daily. Within three months they both started to notice improvements, particularly in respect of their movement disorder and mood.

Skin Disorders

One-hundred-and-ninety patients with eczema or psoriasis had been treated with capsules according to Method 4 by the end of August 2004, at an average dose of four capsules daily. One-hundred-and seventy-six had shown significant clinical improvements in their skin condition within 4 months. This has been evident on examination of the skin, and also in terms of reduced pruritus.

Rheumatoid Arthritis and Osteoarthritis

One-hundred-and-forty-seven patients with arthritis had been treated with capsules according to Method 4 at a dose of between six and eight capsules daily by the end of August 2004. One-hundred-and-twenty-three showed significant clinical improvements within 4 months, including improvements in the range of joint movement and reduced joint pain.

1-46. (canceled)

47. A formulation comprising:
(a) eicosapentaenoic acid or an ester thereof,
(b) a triterpene or an ester thereof, and
(c) substantially no docosahexaenoic acid.

48. A formulation according to claim 47 comprising no docosahexaenoic acid.

49. A formulation according to claim 47 in which the eicosapentaenoic acid or ester thereof is selected from the group comprising natural eicosapentaenoic acids, synthetic eicosapentaenoic acids, naturally occurring esters of eicosapentaenoic acids, synthetic esters of eicosapentaenoic acids, and combinations thereof.

50. A formulation according to claim 47 in which the eicosapentaenoic acid or ester thereof is isolated from fish oil.

51. A formulation according to claim 47 in which the eicosapentaenoic acid or ester thereof is pure.

52. A formulation according to claim 47 in which the triterpene or ester thereof is selected from the group comprising naturally occurring triterpenes, synthetic triterpenes, naturally occurring esters of a triterpene, and synthetic esters of a triterpene, and combinations thereof.

53. A formulation according to claim 52 in which the triterpene is selected from the group comprising 3-O-trans caffeoyl derivatives of betulinic acid, morolic acid or oleanolic acid, faradiol-O-laurate, faradiol-O-palmitate and faradiol-O-myristate.

54. A formulation according to claim 47 in which the triterpene is isolated from the flower heads of marigolds (Calendula officinalis), Zygophyllum eichwaldii, Carthamus lanatus, Oenothera bienni (evening primrose) or Pyrus comminus.

55. A formulation according to claim 54 in which the triterpene is provided in the form of evening primrose oil.

56. A formulation according to claim 55 in which the evening primrose oil is virgin evening primrose oil.

57. A formulation according to claim 47 comprising up to 99% w/w of eicosapentaenoic acid or an ester thereof.

58. A formulation according to claim 47 comprising up to 99% w/w of triterpene or an ester thereof.

59. A formulation according to claim 57 comprising up to 70% w/w of eicosapentaenoic acid or an ester thereof and from 1 to 30% w/w of a triterpene or an ester thereof.

60. A formulation according to claim 47 in a pharmaceutically or cosmetically acceptable form.

61. A formulation according to claim 47 comprising a pharmaceutical or cosmetic carrier, diluent or excipient.

62. A formulation according to claim 47 comprising one or more compounds for co-administration.

63. A formulation according to claim 62 comprising gamma-linolenic acid.

64. A formulation according to claim 62 comprising conjugated linoleic acid.

65. A liquid, a paste, a tablet or a capsule for oral administration, or a compound for topical administration, wherein the liquid, paste, tablet capsule or compound comprises:
(a) eicosapentaenoic acid or an ester thereof;
(b) a triterpene or an ester thereof; and
(c) substantially no docosahexaenoic acid.

66. A method of cosmetic treatment, or a method for the treatment of physiological or disease states including rheumatoid arthritis, osteoarthritis, back-ache, psoriasis, pre-menstrual syndrome, bacterial infections, viral infections, fatigue, insomnia, anxiety, obesity, influenza, diabetes mellitus, alcoholism, cancer, neurological disorders, epilepsy, tardive dyskinesia and choreiform disorders, psychiatric disorders, cardiovascular disorders, dermatological disorders, respiratory disorders, learning disability and ageing, in a subject, the method comprising administering to the subject an effective amount of a formulation comprising:
(a) eicosapentaenoic acid or an ester thereof;
(b) a triterpene or an ester thereof; and
(c) substantially no docosahexaenoic acid.

67. The method of claim 66, wherein the eicosapentaenoic acid, or an ester thereof, and the triterpene, or an ester thereof, are administered simultaneously, either in the same or different formulations, or sequentially.
